(19) United States
(12) Patent Application Publication
(10) Pub. No.: US 2008/0248074 A1
(43) Pub. Date: Oct. 9, 2008

(54) Treatment for Topical Use in Alleviating or Inhibiting Joint Pain or Muscle Pain or Musculoskeletal Pain in a Mammal

(76) Inventor: Donald E. Scott, Rushville, OH (US)

Correspondence Address: WOOD, HERRON & EVANS, LLP, 2700 CAREW TOWER, 441 VINE STREET, CINCINNATI, OH, 45202, US

(21) Appl. No.: 11/815,346

(22) PCT Filed: Feb. 10, 2006
(86) PCT No.: PCT/US06/04760

§ 371 Date: Aug. 2, 2007

Related U.S. Application Data
(60) Provisional application No. 60/652,329, filed on Feb. 11, 2005.

(51) Int. Cl. A61K 9/00; A61K 36/00; A61F 13/49

(52) U.S. Cl. 424/402; 424/774; 604/358

(57) ABSTRACT

A treatment for topical use in alleviating or inhibiting joint pain, muscle pain, or musculoskeletal pain in a mammal. The treatment includes dried nettle plant applied to the skin of a mammal, or includes liquid nettle extract applied to the skin of a mammal, or includes a textile fabric (24) and a treatment composition (32) including an extract of a nettle plant. A method of alleviating or inhibiting joint pain, muscle pain, or musculoskeletal pain in a mammal includes the step of topically applying dried nettle, liquid nettle extract, or a treatment fabric to an area of the skin of the mammal proximate a pain site. A method of making a treatment fabric includes: contacting a liquid with a textile fabric (24), the liquid including an extract (22) of nettle plant; and removing at least a portion of the liquid, thereby forming the treatment fabric.

### CROSS-REFERENCE TO RELATED APPLICATIONS

This application claims the benefit of U.S. Provisional Patent Application Ser. No. 60/652,329, filed Feb. 11, 2005, entitled “Treatment Fabric for Topical Use in Alleviating or Inhibiting Joint Pain or Muscle Pain or Musculoskeletal Pain in a Mammal,” which is incorporated by reference herein in its entirety.

### FIELD OF THE INVENTION

The present invention pertains generally to a composition and method for alleviating pain and, more particularly, to a composition and method in which dried stinging nettle, or an extract thereof, is applied to the skin proximate a pain site to alleviate pain associated with various inflammatory conditions, or other pain conditions.

### Description of Related Art

Many modern medications have been developed from ancient healing traditions associated with specific plants. The medicinal properties of plants have been identified with specific chemical compounds which have been isolated, purified and, in many cases, synthetically reproduced. Many well-known drugs were originally derived from plants. For example, salicylic acid, the precursor for aspirin, was originally isolated from white willow bark and the meadowsweet plant. Quinine, which is used to treat malaria, was derived from Cinchona bark. Morphine, derived from the opium poppy, is still the standard against which new synthetic pain relief drugs are measured.

Modern physicians tend to rely on treatments using synthetic or chemically manufactured drugs. Rather than using whole plants or plant extracts for treatment, pharmacologists tend to identify, isolate, extract, insulate, and synthesize the active compounds from plants for use in treatment. This approach, however, has drawbacks. In addition to the individual physiologically active compounds present in a plant, there are also minerals, vitamins, oils, alkaloids, and other substances which can be important in supporting the medicinal properties of a particular plant. These additional substances can provide a synergistic effect which is absent when purified or synthetic active compounds are used alone.

Various new and old drugs, from aspirin to non-steroidal anti-inflammatory drugs (NSAIDs) to cortisone, have been developed for the treatment of pain associated with inflammation. Musculoskeletal pain, commonly caused by inflammation following injury, is a common reason for self-treatment and/or consultation with a physician. Drugs such as aspirin or NSAIDs are the most common treatment. Arthritis is a general term for a disease involving inflammation of a joint or joints, and encompasses more than one hundred different diseases, frequently having entirely different causes. Osteoarthritis and rheumatoid arthritis, the two most common forms of arthritis, have the greatest public health implications. Osteoarthritis, also known as “degenerative joint disease” or “wear and tear” arthritis, results from physical changes in joints and surrounding tissues, leading to pain, tenderness, swelling, and decreased function. The joints most often affected are the hip, knee, and hand. Rheumatoid arthritis is an autoimmune inflammatory disease of the whole body, characterized by chronic inflammation of the joint linings, not of the joints alone, and in particular, the connective tissues of the body.

In both types of arthritis, many manifestations are similar. The joints, whether singly or in multiples, may become swollen, warm, deformed, gnarled, and in many instances present grotesque deformities. In many cases the adjacent muscles and tendons are affected, as well as other connective tissues of the body, manifested by symptomatic swelling, pain and stiffness. Likewise, musculoskeletal pain, such as pulled muscles and broken bones, and hemorrhoids are characterized by symptomatic inflammation, swelling and pain.

The enormous consumption of painkillers and anti-inflammatory drugs to treat inflammatory conditions such as arthritis, musculoskeletal pain, and hemorrhoids often has undesirable long-term effects, and many of these systemic drugs have dangerous side effects. Their dosage must be carefully prescribed and administered under controlled conditions and circumstances

As an alternative to modern anti-inflammatory drugs, the sting of the common stinging nettle has been used to treat inflammation and arthritic pain. Stinging nettle (Urtica dioica) is a perennial, slow-spreading plant that grows from 5 to 10 feet tall. The leaves are coarsely-toothed, with numerous, small bristly stinging hairs over much of the bottom surface of the support structure of the leaf. The hairs also appear on the stalks during later periods of growth. The undersides of the nettle leaves and stems have small needle-like structures. Stinging nettle is a native species throughout Europe, Asia, Africa, Australia, and the Andes Mountains in South America, as well as in North America. It is confined primarily to moist areas along streams, or in deep, rich soils. Urtica dioica also can be found in sunny areas, and in disturbed soils. Also difficult to eradicate, it is primarily a nuisance to recreationists because of its stinging hairs, which are indeed quite painful to the touch. Another type of stinging nettle that may be used is Urtica urens. Also referred to as a dog nettle or false nettle, it is confined primarily to shaded, moist areas along streams, or in deep, rich undisturbed soils.

The Romans in ancient times used the sting of stinging nettle to alleviate joint and muscle pain by urtication (external stinging or flogging). They also did this to keep warm in the winter. Reports have also been found of nettle urtication for the treatment of arthritic pain by the Thompson Indians in Vancouver Island, British Columbia, throughout the British Isles, and in the United States. Nettle Sting of Urtica Dioica for joint pain—an exploratory study of this complementary therapy, Randall C, Meethan K, Randall H, Dobbs f. Comp. Ther. Med 1999;7:125-131. Research by Dr. Colin Randall at the University of Plymouth, U.K. also reports the use of the sting of the common stinging nettle to treat the pain of osteoarthritis and musculoskeletal pain. Randomized controlled trial of nettle sting for treatment of base-of-thumb pain, Randall C, Randall H, Dobbs F, Hutton C, Sanders H. J R Soc Med. 2000: 93(6):305-309.

While stinging nettle are known for alleviating arthritis pain with their sting, it is also known that a particular nettle extract can relieve the pain of skin burning and irritation. U.S. Pat. Nos. 5,854,291 and 5,856,361 by Holt and Laughlin, entitled “Pain Reliever and Method of Use,” disclose a topically-applied capsaicin-based pain reliever for inflammatory conditions in which an ingredient is required to relieve the side-effect of skin burning and irritation caused by capsaicin. This ingredient is selected from either a polyol, a nettle extract, a yarrow extract, a coltsfoot extract, a birch extract, a rosemary extract, a horsetail extract, a ginger extract, a chamomile extract, a comfrey extract, a lavender extract, or a bergamot extract.

Also, the oral ingestion of nettle for the systemic treatment of arthritis is known. Rose et al., in U.S. Pat. Nos. 5,916,565 and 6,344,220, entitled “Product and Method for Treating Joint Disorders in Vertebrates,” disclose an orally-administered composition including metabolic precursors, herbal phytochemicals, and palatability agents capable of prophylaxis and therapy of joint and connective tissue disorders in vertebrates. The composition is primarily intended for ingestion by dogs, horses, and cats. The herbal phytochemicals are intended to provide a synergistic effect with the metabolic precursors glucosamine and chondroitin sulfate, and include cayenne, ginger, turmeric, yucca, Devil's claw, nettle leaf, Black Cohosh, alfalfa and celery seeds.

While the above uses of nettle may be sufficient for their specific, intended purposes, they each have disadvantages. Accordingly, there remains a substantial need for an effective externally-applied treatment for pain associated with arthritis and other forms of inflammatory disease. There is also a need for such a treatment whereby stinging nettle can be applied to the skin without causing a sting, pain, or rash.

### SUMMARY OF THE INVENTION

Accordingly, it is an object of the present invention to provide an alternative methodology for effectively relieving or inhibiting the pain associated with inflammatory conditions such as arthritic inflammation, musculoskeletal inflammation, and/or hemorrhoids, as well as other swelling, injury, or elevated temperature. Another objective of the present invention is to provide a method for applying dried nettle, or an extract thereof, to a pain site. It is another objective to provide a composition including dried stinging nettle, preferably processed in a form in which the nettle no longer causes a sting or rash when applied. It is a further objective to provide a method of processing dried stinging nettle for use in pain relief from inflammation.

To this end, a method of alleviating or inhibiting pain associated with inflammation according to the present invention includes the step of applying dried stinging nettle, or an extract thereof, to the skin proximate a pain site. This may include application directly to a pain site, such as a cut or burn, or application near a pain site, such as on the forehead to treat a headache, or on the scrotum to treat prostate pain. The inflammation may be caused by various physical injuries or conditions, such as, for example, osteoarthritic inflammation, musculoskeletal inflammation, and hemorrhoids. The method described herein is extremely advantageous since, according to a preferred embodiment of the invention, no other medications need be combined with the nettle to alleviate the pain.

As a nonlimiting example of how the invention is used in practice, a person experiencing pain from inflammation may take a dried nettle plant, or preferably a small amount of a dry mixture of component parts thereof, comprised of dried nettle leaves and/or buds, and apply the nettle directly to the skin overlying the painful area. Preferably the applied nettle is then temporarily secured in place after application by a bandage or the like, preferably for a period of at least about 12 hours, and more preferably for at least about 72 hours. When used as a dried form of the nettle, the nettle may be in the form of powder or flakes. The bandage may be in the form of a wrap, such as a stretch wrap for the arms or legs, or may be in a form including an adhesive for application to other parts of the body. Thereafter the nettle is removed and the person may enjoy relief from the pain.

Another nonlimiting example includes an extract of a nettle plant, such as Urtica dioica. Thus, one aspect of the invention is directed to a treatment fabric for topical use in alleviating or inhibiting joint pain, muscle pain, or musculoskeletal pain in a mammal, such as from swelling, injury, or elevated temperature, for example. The treatment fabric includes a textile fabric and a treatment composition including an extract of Urtica dioica.

Another aspect of this nonlimiting example of the invention is directed to a method of alleviating or inhibiting joint pain, muscle pain, or musculoskeletal pain in a mammal. The method includes the step of topically applying a treatment fabric to an area of the skin of the mammal proximate a pain site. This may include application directly to a pain site, such as a cut or burn, or application near a pain site, such as on the forehead to treat a headache, or on the scrotum to treat prostate pain. The treatment fabric includes a textile fabric and a treatment composition including an extract of Urtica dioica.

A further aspect of this nonlimiting example of the invention is directed to a method of making a treatment fabric for topical use in alleviating or inhibiting joint pain, muscle pain, or musculoskeletal pain in a mammal. The method includes: contacting a liquid with a textile fabric, the liquid including an Urtica dioica extract; and removing at least a portion of the liquid, thereby forming a treatment fabric comprising the textile fabric and an extract of Urtica dioica.

Another nonlimiting example includes the direct application of an extract of a nettle plant, such as Urtica dioica, to the skin of a mammal, for use in alleviating or inhibiting joint pain, muscle pain, or musculoskeletal pain in the mammal.

A further understanding of the nature and advantages of the present invention will become apparent when taken together with the remaining portions of the specification.

### BRIEF DESCRIPTION OF THE DRAWINGS

FIG. 1 is a schematic of an extraction process in accordance with the principles of the present invention.

FIG. 2 is a cross-section of a treatment fabric impregnated with nettle extract, in accordance with the principles of the present invention.

FIG. 3 is a cross-section of a bandage including a treatment fabric impregnated with nettle extract, in accordance with the principles of the present invention.

### DETAILED DESCRIPTION OF SPECIFIC ASPECTS OF THE INVENTION

Definitions: The following terms used herein are intended to have the following meanings:

Cellular integrity as it applies to stinging nettle is defined as the microscopic, cellular makeup being intact, unimpaired and/or free from injury. Actions such as freezing, pulverizing or crushing may destroy cellular integrity.

Component Dart is defined as any one or more of bud, leaf, stem (stalk), root, and seed of a stinging nettle plant. The terms bud, leaf, stem, root, and seed can mean either the singular or the plural.

Dried or Drying is defined as the physical state of a harvested stinging nettle plant in which the plant no longer has the ability to cause the notoriously painful sting associated with stinging nettle, due to the plant becoming wizened, shriveled, wrinkled and/or shrunken as a result of a loss of natural moisture and vitality. Once the leaves or other component parts of the nettle plant have been harvested, the hairs begin to wilt almost immediately.

Harvested as it relates to a nettle plant is defined as gathered, removed, or extracted from a growing medium, such as the ground or another portion of the plant.

Inflammation is defined as a local response to a physiological condition which typically results in pain, and may be accompanied by redness, swelling and/or heat.

Maturity or mature as it relates to stinging nettle is defined as that which has been allowed to grow long after blossoming, and has been harvested as late into the fall season as possible, before a freeze.

Milling as it relates to processing of stinging nettle is defined as mixing and mingling, preferably manually, of one or more component parts of a dried nettle plant or plants without destroying the cellular integrity of the component part(s).

Nettle is defined as a plant, multiple plants, and/or one or more portions of the plant(s) belonging to a strain of stinging nettle. The particular strain of stinging nettle plant includes, but is not limited to, the family Urtica dioica and Urtica urens. Urtica dioica, also known as Urtica gracilis, is a hardy, perennial plant with slender leaves that grows up to 5-10 feet tall. Urtica urens is a annual, greener, smaller variety of stinging nettle with broader leaves, and grows up to 2-5 feet tall.

Pain site is defined as the area or location on the body of an individual experiencing pain associated with inflammation. The pain may be caused by injury, swelling, and/or elevated temperature, among others. These pain sources are merely exemplary.

Portion is defined as any part of the nettle plant, ranging from a small part of one of the component parts to the entire plant.

The invention is directed to a method to alleviate pain associated with various conditions such as, for example, osteoarthritic inflammation, musculoskeletal inflammation, and hemorrhoids, as well as other injury, swelling, or elevated temperature. In one aspect, the method includes the application of dried nettle to the skin overlying a pain site. This may include application directly to a pain site, such as a cut or burn, or application near a pain site, such as on the forehead to treat a headache, or on the scrotum to treat prostate pain. Preferably the dried nettles are exclusively a combination of the leaves and buds of the plant, but other component parts such as the seeds or stems may be applied as well. However, no other medications need be combined with the nettle. The nettle may be kept in place by a bandage or the like, preferably for a period of at least about 12 hours, and more preferably for at least about 72 hours. While not being bound by any theory, it is believed that, when used in this manner, the applied dried nettle reduces swelling and inflammation associated with pain, and does so for a relatively long period of time, as compared to aspirin, NSAIDs, steroid injections, or other conventional medications or treatments. Nettles are painkillers and diuretics, and attract the components and effects of the immune system to the area to which they are applied. As nettles as diuretics, following treatment, it may be beneficial to rehydrate the skin of the mammal at the area of treatment.

Another aspect of the invention is directed to a treatment fabric for topical use in alleviating or inhibiting joint pain, muscle pain, or musculoskeletal pain, for example, in a mammal. The treatment fabric includes a textile fabric and a treatment composition including an extract of a nettle plant, such as Urtica dioica. Another aspect of the invention is directed to a method of alleviating or inhibiting joint pain, muscle pain, or musculoskeletal pain in a mammal. The method includes the step of topically applying a treatment fabric to an area of the skin of the mammal proximate a pain site. The treatment fabric includes a textile fabric and a treatment composition including an extract of Urtica dioica. A further aspect of the invention is directed to a method of making a treatment fabric for topical use in alleviating or inhibiting joint pain, muscle pain, or musculoskeletal pain in a mammal. The method includes: contacting a liquid with a textile fabric, the liquid including an Urtica dioica extract; and removing at least a portion of the liquid, thereby forming a treatment fabric comprising the textile fabric and an extract of Urtica dioica. While the use of Urtica dioica is described above and elsewhere, it will be recognized by those skilled in the art that other stinging nettle plants, such as Urtica urens, may be used.

The same treatment fabric, made in the same way, also may be used in the same fashion to alleviate and/or inhibit one or more symptoms associated with the following additional conditions: prostate enlargement; headaches; inter-vertebral disk inflammation; acid reflux; burns; Barrett's Disease; pleurisy; lacerations; fibromyalgia; warts; tumors; bone spurs; and surgeries, such as implanted knees and hips, and oral surgeries, such as to treat tooth abscesses. In addition, the treatment fabric and method may be used to alleviate and/or inhibit inflammation, as well as to alleviate and/or inhibit one or more symptoms of conditions in which inflammation is implicated or involved.

Another aspect of the invention is directed to a treatment for topical use in alleviating or inhibiting joint pain, muscle pain, or musculoskeletal pain, for example, in a mammal. This aspect includes a treatment composition including an extract of a nettle plant, such as Urtica dioica. The method of using this composition includes the step of topically applying the composition to an area of the skin of the mammal proximate a pain site. Like the treatment fabric described above, nettle plants other than Urtica dioica may be used. The extract may be applied directly to the skin without any vehicle, such as a cream, lotion, ointment, or other vehicle.

Advantageously, Urtica dioica, is grown long after blossom (Urtica dioica typically blossom in mid-summer) before harvesting in late fall, before a freeze. This is because the mature plant may be more potent and have a longer duration of action. However, the leaves at the top of the plant generally have higher concentrations of the ingredients to be extracted, and so these can be used from plants that are less mature. Advantageously, the bottoms of the seedpods have started to turn brown. The plant may then be harvested by cutting at the base of the stalk, pulling by the root, or the like. It may be advantageous to select plants that have been exposed to the sun during a substantial portion of their growth. Further, the plant is shocked by frost (but not killed), and it is killed by a freeze. However, the concentration of nettles increases after a frost. Thus, the nettle plant or parts thereof may be harvested prior to frost (to prevent losing the plant in the event a freeze occurs before a frost); but once a frost occurs, harvesting preferably occurs rapidly.

The plant then may be prepared for alcohol extraction. In this stage, if desired, the Urtica dioica may be dried. Alternatively, the drying step may be omitted, and the plant may be further prepared for alcohol extraction. The plant may be dried using any suitable method. For example, the plant may be hung upside down in a well-ventilated area, such as a barn. Further, it is believed that hanging in this manner may allow one or more active ingredients which may be in the stalk and/or roots to move into the leaves and/or buds. Alternatively, moisture may be removed from the harvested plant by circulating air around the plant. For example, if desired, large air-moving fans may be used to circulate air in the location where moisture is being removed from the harvested plants. Advantageously, the plants may be interned or rotated every few hours to assist the moisture-reducing process. Alternatively, the moisture-reducing step described above may be omitted.

If desired, the entire harvested Urtica dioica plant may be subjected to alcohol extraction, with the alcohol extraction step described in further detail below. Alternatively, the plant may be processed so as to exclude one or more component parts of the plant, or increase the relative percentage of one or more particular component parts to be subsequently extracted. For example, if desired, the entire harvested plant may be used in the alcohol extraction. Alternatively, stems and large stalks may be removed. Also, if desired, plant parts that appear brown in color may be removed. Alternatively, the plant may be processed so that the vast majority, if not all, of the plant parts subjected extraction are predominately-green buds, seeds, bud “dust”, and/or seed husks. In another aspect, the plant may be processed so that the majority, if not all, of the component parts subjected to the extraction step are Urtica dioica leaves and/or buds.

After drying, the nettle plant is preferably milled in order to convert, break apart, separate and/or reduce the nettle plant into its component parts. Also, it is preferable to continue to mill the dried nettles prior to extraction. Since it is preferred to apply mainly leaf and bud only over the pain site, it is also preferable to separate out and remove the stems from the milled component parts. Optionally, one may also separate out and remove the seeds. Additionally, it is preferable that large lots of nettle plants are combined prior to the milling step to assure consistency in the potency of the batch.

While not being bound by theory, it is believed to be advantageous to treat the nettle plant in a manner that does not freeze, crush, damage or lyse the cells, or otherwise destroy cellular integrity. This is because the active substance or substances responsible for the pain alleviating effects are believed to be found only in intact cells. In addition, it is believed that these active substance(s) are more potent and have a longer duration of action in the mature plant. Therefore, it may be important during processing of the harvested nettle plant that actions such as freezing or pulverizing not be done, because this may destroy the cellular integrity of the component parts, especially the leaves and buds.

In the extraction stage, any suitable alcohol may be used, with examples including isopropyl alcohol and ethyl alcohol. The harvested Urtica dioica and/or select component plant parts obtained via post-harvest processing as described above, may be placed in any suitable container for alcohol extraction. Typically, the liquid for alcohol extraction is a mixture of alcohol and water, as opposed to 100% alcohol. As used herein, the term “alcohol” is used to describe both 100% alcohol and alcohol-water mixtures. The extraction alcohol may be added to the container before, during, and/or after, the addition of the harvested plant or component part(s). Advantageously, during the extraction process, enough alcohol is present in the container so as to keep the component parts under the surface of the liquid throughout the entire extraction process.

In Urtica dioica in which the moisture content has not been actively reduced (for example, by air drying for a period of time), the extracting alcohol advantageously may have an alcohol content, which is efficient from about 70 to about 91% alcohol by weight, with the remainder being water. Alternatively, if moisture reduced plant material is used, then, if desired, an extracting alcohol having a relatively lower percentage of alcohol in the alcohol/water mixture may be used. For example, such an extraction alcohol, which is efficient, may include from about 50 to about 70% alcohol by weight, with the remaining amount being water. In one particular embodiment, the extraction alcohol may include just over 50% alcohol by weight, for example 51% alcohol by weight, with the remaining amount being water.

The time for extraction also advantageously may be varied depending on the moisture content of the Urtica dioica plant materials in the alcohol extraction container. For example, if the harvested plant materials have been dried so that the moisture-content of the plant is relatively low, then, if desired, the alcohol extraction may be allowed to occur over a period of about three days. On the other hand, if freshly harvested plant materials are placed in the extracting liquid (i.e., plant having a relatively high moisture content), then, advantageously, the extraction process may be conducted over a period of many days, and even weeks, with one example being about 21 days.

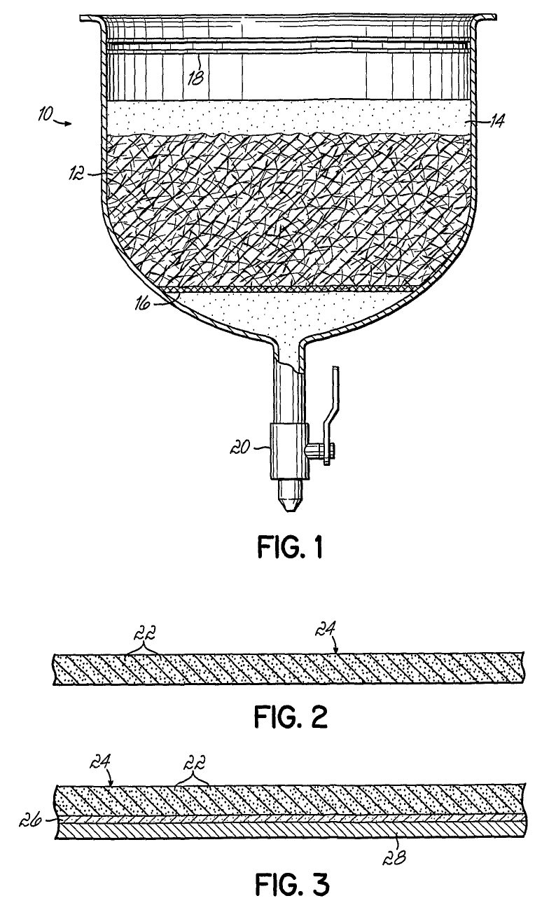

In one particular nonlimiting method, and referring to FIG. 1, the extraction process may be conducted in a tank 10. This tank may be made of any suitable material, such as stainless steel. The nettles 12 and extraction alcohol 14 (such as the approximately 51% alcohol by weight extraction alcohol) may be placed within the tank 10. A screen 16 may be placed near the bottom of the tank 10 for the nettles 12 to rest on. This screen 16 allows the nettles 12 to be immersed in the extraction alcohol 14, but prevents them from settling to the bottom of the tank 10. A vapor barrier 18 may be placed above the extraction alcohol 14, and within the tank 10, to prevent the evaporation of alcohol. The tank 10 may be kept at any temperature suitable for the extraction process. In one embodiment, this temperature may be under 50° F. The tank 10 may also be impermeable to light. And the tank 10 may include a valve 20 to remove liquid from the tank 10, once the extraction process is complete. That liquid may be processed into a usable form, or alternatively may be saved and mixed with further liquid from a second extraction (using the same or different nettles). This mixing of multiple extractions may also be done to enhance consistency of the concentration of nettle extract. In removing the liquid from the tank, the initial extract may flow from the valve as a “sludge.” This “sludge” may be discarded prior to using the liquid extract as a treatment composition, or to apply to cloths to form a treatment fabric.

The Urtica dioica extract liquid then may be further processed using any suitable equipment and technique. The particular equipment and/or method(s) generally correspond with the ultimately desired physical form of the plant extract, something which depends in large part on the desired finished product. For example, the plant extract may be kept in a liquid form, or the liquid may be removed, leaving behind a dry plant extract. In either case, if desired, the plant extract may be combined with any of a number different active and/or inert ingredients depending upon the particular end product, and physical form, desired. For example, the plant extract may be formulated for topical administration as a liquid, a cream, or an ointment.

Alternatively, the Urtica dioica extract liquid may be brought into contact with a textile fabric, and the alcohol and water may be removed using any of a number of suitable techniques. The textile fabric, itself, may be any suitable fabric. Examples include wovens, knits, felts, and paper (such as would be used to form paper towels). Advantageously, the textile fabric may be woven clothing made of all-natural yarns, for example, all-cotton yarns. If desired, commercially available diaper cloth may be used.

In one exemplary set-up, the suitablely sized length of an all-cotton woven cloth may be laid out on the bottom surface of a container (for example, a tray). An amount of Urtica dioica extract liquid then may be poured into the container, thereby allowing the plant extract liquid to may absorbed by the cloth. Alternatively, the Urtica dioica extract liquid may be sprayed onto the cloth. Depending on the production volume desired, many containers (i.e., trays), each containing a length of clothing and plant extract liquid may be processed simultaneously. If desired, forced air circulation may be used to assist in the evaporation of the liquid for a period of time, for example, from about four to eight hours. Then, when most of the liquid has evaporated, forced air circulation may come if desired, be continued at a lower airflow rate. During this evaporation process, advantageously, the containers are not exposed to direct sunlight. Referring to FIG. 2, as the liquid evaporates, the plant extract 22 is absorbed into, absorbed onto, and/or mechanically entangled with the fibers of the particular fabric cloth 24. Often times, and preferably, the fabric will have a rich deep green color, due to the Urtica dioica extract. After the extracting liquid has evaporated, and the textile fabric has dried, the fabric may be further processed as desired. Typically, the drying process takes from about three to about four days. However, this length of time depends on various factors, including, for example, the relative humidity. Thus, apparatus such as dehumidifiers may be used.

During the drying process, the nettle extract generally migrates to the top surface of the cloths. Thus, that side of the cloth exhibits a higher concentration of nettle extract than the opposite side. As a result, the top side of the cloth may be placed in direct contact with the skin of a mammal for a “heavy” treatment. Alternatively, the opposite side of the cloth may be placed in direct contact with the skin of a mammal for a “light” treatment.

At this point, the Urtica dioica extract fabric may be used for topical administration. Alternatively, the extract-containing fabric may be saved for future use. If that is the case, the fabric may be stored in any suitable fashion. For example, the fabric may be placed in a sealed air-resistant bag and frozen. The fabric may also be kept from exposure to light, and/or preservatives may be added to the fabric.

Depending upon the particular condition for which the treatment fabric is being used, and/or the particular location of the area of skin to which the fabric is applied, it may be beneficial to cut the treatment fabric into one of any of a number of different shapes and sizes. Also, and referring to FIG. 3, it may be helpful to attach a piece of the treatment fabric 24 to an adhesive strip 26 or patch, or a length of elasticized bandage-wrap material. The bandage may assist in releasably holding the piece of treatment fabric against the skin. A support sheet 28, such as wax paper, may be used to cover the adhesive 26 until the fabric 24 is used. One then can remove the wax paper and apply the adhesive directly to the skin. Alternatively, one can apply the adhesive side to the interior of clothing, such that when the clothing is worn, the treatment fabric contacts the user's skin proximate to a pain site.

In use, the treatment fabric typically is worn on the area of the skin for a treatment period of from about 24 to about 72 hours, after which the treatment fabric may be removed. Once the fabric is removed, if the skin area itches or is otherwise irritated, then the area may be cleaned with isopropyl alcohol, followed by water.

It is to be understood that the above description is intended to be illustrative and not restrictive. Many embodiments may become apparent to those of ordinary skill in the art upon reviewing the above description. By way of example, the invention has been described primarily with reference to the use of bandages or wraps to keep the nettle in place. However, it may be readily recognized that other items could be used to maintain the nettle in contact with the skin. The scope of the invention should, therefore, be determined not with reference to the above description, but instead be determined with reference to the appended claims, along with the full scope of equivalents to which such claims are entitled.

What is claimed is

1. A treatment fabric for topical use in alleviating or inhibiting joint pain or muscle pain or musculoskeletal pain in a mammal, the treatment fabric comprising:
a textile fabric; and
a treatment composition including an extract of nettle.

2. The treatment fabric of claim 1 wherein the treatment composition consists essentially of an extract of nettle, the nettle being Urtica dioica.

3. The treatment fabric of claim 1 wherein the textile fabric consists essentially of all-natural fibers.

4. The treatment fabric of claim 1 wherein the textile fabric is a woven fabric.

5. The treatment fabric of claim 1 wherein the textile fabric is diaper cloth.

6. A method of alleviating or inhibiting joint pain or muscle pain or musculoskeletal pain in a mammal, the method comprising:
topically applying a treatment fabric to an area of the skin of the mammal proximate a pain site,
the treatment fabric comprising:
a textile fabric; and
a treatment composition including an extract of nettle.

7. The method of claim 6 wherein the treatment composition consists essentially of an extract of nettle, the nettle being Urtica dioica.

8. The method of claim 6 wherein the textile fabric consists essentially of all-natural fibers.

9. The method of claim 6 further including the step of wearing the treatment fabric on the area of the skin for a treatment period of from about 24 to about 72 hours.

10. The method of claim 9 further including the steps of removing the treatment fabric after the treatment period, applying alcohol to the area of the skin, and applying water to the area of the skin.

11. A method of making a treatment fabric for topical use in alleviating or inhibiting joint pain or muscle pain or musculoskeletal pain in a mammal, the method comprising:
contacting a liquid with a textile fabric, the liquid including an extract of nettle; and
removing at least a portion of the liquid, thereby forming a treatment fabric comprising the textile fabric and the extract of nettle.

12. The method of claim 11 further including the step of making an extract of nettle, the nettle being Urtica dioica.

13. The method of claim 12 wherein the making step includes performing an alcohol extraction of Urtica dioica.

14. The method of claim 13 wherein the alcohol extraction is performed using an alcohol/water mixture, in which the alcohol is present in an amount of from about 50 to about 90% by weight.

15. The method of claim 13 wherein the alcohol extraction is performed using an alcohol/water mixture, in which the alcohol is present in an amount of about 51% by weight.

16. A treatment composition for topical use in alleviating or inhibiting joint pain or muscle pain or musculoskeletal pain in a mammal, the treatment composition comprising an extract of nettle.

17. The treatment composition of claim 16 consisting essentially of an extract of Urtica dioica.

18. The treatment composition of claim 16 wherein the extract of nettle is in dried form.

19. The treatment composition of claim 16 wherein the extract of nettle is in the form of a liquid including the extract of nettle.

20. A method of alleviating or inhibiting joint pain or muscle pain or musculoskeletal pain in a mammal, the method comprising:
topically applying a treatment composition to an area of the skin of the mammal proximate a pain site;
the treatment composition comprising an extract of nettle.

21. The method of claim 20 consisting essentially of an extract of Urtica dioica.

22. The method of claim 20 wherein the extract of nettle is in dried form.

23. The method of claim 20 wherein the extract of nettle is in the form of a liquid including the extract of nettle.
